(19) United States
(12) Patent Application Publication
(10) Pub. No.: US 2010/0179417 A1
(43) Pub. Date: Jul. 15, 2010

(54) MEDICAL TUBE SYSTEM WITH POSITION SENSING

(76) Inventor: Susan Russo, Lincoln City, OR (US)

Correspondence Address: Silicon Forest Patent Group;Paul J. Fordenbacher, 11876 NW Tyler Ct, Portland, OR, 97229, US

(21) Appl. No.: 11/993,218

(22) PCT Filed: Jun. 20, 2006
(86) PCT No.: PCT/US06/23974

§ 371 Date: Dec. 20, 2007

Related U.S. Application Data
(60) Provisional application No. 60/692,530, filed on Jun. 20, 2005.
(60) Provisional application No. 60/747,091, filed on May 11, 2006.

(51) Int. Cl. A61B 6/00; G08B 23/00; A61M 39/00

(52) U.S. Cl. 600/424; 340/573.1; 604/264

(57) ABSTRACT

A system including a position-sensing medical tube and sensor electronics is provided in accordance with an embodiment of the present invention. The position-sensing medical tube comprises a medical tube and position-sensor apparatus coupled to the medical tube in a predetermined location. The position-sensor apparatus is adapted to communicate with the sensor electronics so as to provide information dependent on the relative position of the medical tube. Movement of the medical tube, and thus the coupled position-sensor apparatus is sensed by the sensor electronics. The sensor electronics interprets state-data as that the medical tube has moved and responds in a predetermined way, such as, but not limited to, triggering an alarm and turning off a process.

### RELATED APPLICATIONS

This is a non-provisional application claiming benefit under 35 USC §119(e) to U.S. Provisional application Nos. 60/692,530, filed on Jun. 20, 2005, and 60/747,091, filed on May 11, 2006, which are in their entireties incorporated herewith by reference.

### FIELD OF THE INVENTION

The present invention is related to medical equipment, and more particularly, to methods and apparatus for medical tubes.

### BACKGROUND

Many medical procedures involve the use of medical tubes having one end located outside of the patient's body and the other end located inside of the body. Examples of such medical tubes include, but are not limited to, orogastric tubes, nasogastric tubes, percutaneous endoscopic gastrostomy tubes, percutaneous endoscopic jejunostomy tubes, endotracheal tubes, chest tubes, urinary catheters, intravenous catheters, arterial catheters, gastric decompression tubes, various suction tubes, various drainage tubes and intracranial pressure monitors.

Virtually any medical tube device which is associated with a patient for an extended period of time, such as, but not limited to, for the administration of medicine, food or oxygen, the performance of a medical procedure, or the drainage of fluids, can be removed or displaced inadvertently by the caregiver or patient. Removal or displacement of such tubes can be particularly serious, causing morbidity or even mortality, and increase the risk of medical malpractice litigation.

In one example, a nasogastric tube is used to provide enteral nutritional support to a patient with a functional gastrointestinal tract who cannot meet their caloric needs by taking in foods orally. The nasogastric tube is commonly constructed of a flexible material, such as, but not limited to polyvinyl chloride. The nasogastric tube has a tube proximal end, a tube distal end, and at least one lumen there between. There are several nasogastric tubes available on the market for administering enteral nutrition to patients which vary in length, composition, diameter and number of lumens.

The nasogastric tube is inserted through the nose or mouth of the patient and advanced into the stomach or duodenum. The distal end of the nasogastric tube that resides in the stomach or duodenum has apertures through the walls of the tube that allow the enteral nutrition to exit the lumen and enter the patient. The proximal end of the nasogastric tube that resides outside of the body is connected to one of several devices that allow for the administration of enteral nutrition into the lumen and thus into the patient.

A syringe can be connected to the proximal end of the nasogastric tube to give a bolus of enteral nutrition. The enteral nutrition can also be placed into a reservoir bag that is connected to a tube that couples to the proximal end of the nasogastric tube. This allows the enteral nutrition to travel from the reservoir bag to the patient by the use of gravity. The tube of the reservoir bag can also pass through a feeding pump that allows for the delivery of the enteral nutrition to the patient at a specific volume per hour.

Enteral tube feeding has been proven to promote nitrogen retention, accelerate wound healing, and improve overall nutritional status. Enteral tube feeding is favored over intravenous feeding because it helps to maintain intestinal integrity and has a lower infection risk. One of the major drawbacks of enteral tube feeding, however, is the possibility of aspiration of gastric contents into the lungs.

Aspiration is one of the most serious and potentially life-threatening complications of enteral tube feeding. This complication is documented to occur in nearly one percent (0.8%) of the patients receiving a course of enteral nutrition. Aspiration is the condition wherein the enteral nutrition inadvertently enters the esophagus and then subsequently into the lungs. A primary cause of aspiration is when the nasogastric tube becomes displaced and the distal end of the nasogastric tube becomes malpositioned in the esophagus. The enteral nutrition is then delivered directly into the esophagus and subsequently into the lungs.

The consequences of enteral nutrition entering the lungs can range from coughing and wheezing to infection and respiratory failure. The effect of aspiration on the patients depends on the volume, pH, particle size, composition and microbial content, among others, of the aspirated material and the health of the patient. In addition to the possible human suffering incurred with such a complication, expenses on the order of thousands of dollars per event per day can be generated by antibiotic costs, intensive care and respiratory support.

There are many protocols used in the clinical environment aimed at preventing aspiration. These include surveillance of nasogastric tube placement, monitoring gastrointestinal residual volume, elevating the head of the bed, using medications to enhance gastric emptying, using smaller diameter nasogastric tubes, postpyloric placement of nasogastric tubes, securing the nasogastric tube to the patient, and restricting the use of patient's hands to prevent displacement of the nasogastric tube. Nevertheless, aspiration still occurs in patients receiving enteral nutrition with currently-available nasogastric tubes.

Despite the best efforts to address the problems of displacement of nasogastric tubes and aspiration, the solutions to date have been ineffective for the most part or potentially injurious. Moreover, complicated and impractical “solutions” annoy the medical staff, generate extra costs, and place patients at risk. The current trend in medicine towards managed care will put pressure on hospitals to reduce complication rates while keeping costs down. Cost-cutting measures lead to leaner staffing and therefore, less supervision of patients with nasogastric tubes. Not only will displacements of nasogastric tubes increase in this setting but the discovery of displacement will be protracted making aspiration more likely and patient morbidity more severe.

Methods and apparatus for the detection of medical tube displacement are needed in the art to provide opportunity for early intervention in order to thwart potential medical complications. The detection should be easily sensed by the medical staff or by automated systems to allow for remedial measures to be taken so that the associated morbidity and mortality can be prevented. The methods and apparatus should be readily acceptable and easy to use by the medical staff, safe for the patient, and inexpensive to manufacture.

### SUMMARY

The present invention is related to detecting the displacement of medical tubes within the body. It is understood that the term “medical tubes” is used in a general sense and include those tubes having one end internal and one end external to the body. Examples of these medical tubes include, but are not limited to, orogastric tubes, nasogastric tubes, percutaneous endoscopic gastrostomy tube, percutaneous jejunostomy tubes, endotracheal tubes, chest tubes, urinary catheters, intravenous catheters, arterial catheters, gastric decompression tubes, suction tubes, drainage tubes, and intracranial pressure monitors.

A system including a position-sensing medical tube and sensor electronics is provided in accordance with an embodiment of the present invention. The position-sensing medical tube comprises a medical tube and position-sensor apparatus coupled to the medical tube in a predetermined location. The position-sensor apparatus is adapted to communicate with the sensor electronics so as to provide information dependent on the relative position of the medical tube. Movement of the medical tube, and thus the coupled position-sensor apparatus is sensed by the sensor electronics. The sensor electronics interprets state-data as that the medical tube has moved and responds in a predetermined way, such as, but not limited to, triggering an alarm and turning off a process.

In accordance with an embodiment of the present invention, a medical tube system is provided comprising one or more radio-frequency identification (RFID) tags coupled to a medical tube, and associated electronics adapted to detect the RFID tags. The associated electronics includes an RFID sensor element, data communication electronics, and receiving electronics. Each of the RFID tags is coupled to the medical tube in a predetermined location on the tube distal portion. When the tube distal portion of the medical tube is properly positioned within the patient's body, the RFID tag is located within the patient's body. The RFID sensor element is placed on a relatively stationary part of the patient. When the medical tube becomes displaced from its proper placement, the RFID tag moves relative to the RFID sensor element. This movement of the RFID tag is sensed by the RFID sensor element, which in turn communicates state-data to the data communication electronics, and to the receiving electronics. The receiving electronics interprets the state-data and commences an event, such as, but not limited to, notifying the operator and/or terminating a process for which the medical tube is being used in response to the medical tube having moved in relationship to the patient.

In accordance with an embodiment of the present invention, a medical tube system is provided comprising an optical-tagged medical tube comprising a medical tube, and wherein the position-sensor apparatus is a light sensor apparatus coupled thereto, and the sensor electronics is sensor electronics. The light sensor apparatus is adapted to detect light and transmit a signal based on the intensity of the light to the sensor electronics. The system operates on the premise that the amount of available light is greater outside than inside a patient's body. When the optical-tagged medical tube is properly placed within the patient's body, the light sensor apparatus detects a first intensity of light, whereas when the optical-tagged medical tube is displaced from the proper position, the light sensor apparatus detects an increased intensity of light. Sensor electronics is adapted to detect the intensity of the light received by the light sensor apparatus and provide a response suitable for a particular purpose. The light that is detected by the light sensor apparatus may come from one or more sources outside of the patient, such as, but not limited to, ambient room light and a dedicated light source.

Embodiments of a medical tube system of the present invention provide a medical tube system that continuously monitors whether it has become displaced which could potentially lead to clinical problems, such as aspiration into the patient's lungs associated with using a nasogastric feeding tube, by way of example. The displacement of the feeding tube is made apparent to medical staff and automated systems are provided to shut off the delivery of enteral nutrition to the patient. This allows remedial measures to be taken so that the associated morbidity and mortality can be prevented. The methods and apparatus are readily acceptable and easy to use by the medical staff, safe for the patient, and inexpensive to manufacture. Other embodiments are as presented herein.

### BRIEF DESCRIPTION OF THE DRAWINGS

Like reference numbers generally indicate corresponding elements in the figures.

FIG. 1 is a side perspective view of a system including an RFID-tagged medical tube comprising a medical tube and a radio-frequency sensor apparatus, in accordance with an embodiment of the present invention;

FIG. 2 is a schematic diagram of the radio-frequency sensor apparatus comprising one or more radio-frequency identification (RFID) tags, an RFID sensor element, data communication electronics, and receiving electronics, in accordance with an embodiment of the present invention;

FIG. 3 is a front partial cut-away view of a medical tube system comprising an RFID-tagged medical tube and an RFID sensor element, in accordance with an embodiment of the present invention;

FIG. 4 is a front partial cut-away view of a medical tube system comprising an RFID-tagged medical tube and an RFID sensor element relative to a patient, in accordance with an embodiment of the present invention;

FIG. 5 is a front partial cut-away view showing a medical tube system comprising an RFID-tagged nasogastric tube and an RFID sensor element relative to a patient, in accordance with an embodiment of the present invention;

FIG. 6 is a front partial cut-away view of a medical tube system comprising an RFID-tagged nasogastric tube and a plurality of RFID sensor elements, in accordance with an embodiment of the present invention;

FIG. 7 is a front partial cut-away view showing a system comprising an RFID-tagged medical tube and a handheld RFID sensor relative to a patient, in accordance with an embodiment of the present invention;

FIG. 8 is a side view of a system including an optical-tagged medical tube comprising a medical tube, light sensor apparatus, and sensor electronics, in accordance with an embodiment of the present invention;

FIG. 9 is a side view of a system including an optical-tagged medical tube comprising a medical tube, light sensor apparatus, and sensor electronics, in accordance with an embodiment of the present invention;

FIG. 10 is a flow chart of a method in accordance with an embodiment of the present invention;

FIG. 11 is a side perspective view wherein the LGs are coupled within a groove extending from the tube outer surface, in accordance with an embodiment of the present invention;

FIG. 12 is a side perspective view wherein the LGs are coupled within a groove extending from the tube outer surface, and the groove is filled in with transparent material to a conformal surface with the tube outer surface, in accordance with an embodiment of the present invention;

FIG. 13 is a side perspective view wherein the LG is embedded in the tube wall such as might be provided in a co-extrusion process, in accordance with an embodiment of the present invention;

FIG. 14 is a side perspective view wherein the medical tube further comprises a sensor lumen that extends from the tube proximal end to the tube distal end, in accordance with an embodiment of the present invention;

FIG. 15 is a cross-sectional view of the medical tube further comprising a sensor lumen that extends from the tube proximal end to the tube distal end, in accordance with an embodiment of the present invention;

FIG. 16A is a cross-sectional view of the medical tube further comprising a sensor lumen that extends from the tube proximal end to the tube distal end, in accordance with an embodiment of the present invention;

FIG. 16B is a top view of a LG sheet, in accordance with an embodiment of the present invention; and

FIG. 17 is a side perspective view of a system including an optical-tagged medical tube comprising a medical tube, light sensor apparatus, and sensor electronics, in accordance with an embodiment of the present invention.

### DETAILED DESCRIPTION

References will now be made to embodiments illustrated in the drawings and specific language which will be used to describe the same. It will nevertheless be understood that no limitation of the scope of the invention is thereby intended, such alterations and further modifications in the illustrated devices, as such further applications of the principles of the invention as illustrated therein as being contemplated as would normally occur to one skilled in the art to which the invention relates.

Embodiments in accordance with the present invention relate to detecting the placement and displacement of medical tubes within a patient's body. It is understood that the term “medical tubes” is used in a general sense and include those tubes adapted to have one end internal and one end external to the body. Examples of medical tubes include, but are not limited to, orogastric tubes, nasogastric tubes, percutaneous endoscopic gastrostomy tubes, percutaneous endoscopic jejunostomy tubes, endotracheal tubes, chest tubes, urinary catheters, intravenous catheters, arterial catheters, gastric decompression tubes, suction tubes, drainage tubes, and intracranial pressure monitors. The description provided below includes reference to nasogastric tubes and detection of nasogastric tube displacement. It is understood that the description below is provided by way of example and is not limited to the described applications.

Medical tubes come in a variety of configurations, including single lumen and multi-lumen configurations. The one or more lumens can extend the entire length of the medical tube, or one or more of the lumens can terminate at a predetermined distance from one of the medical tube ends. For example, there are known nasogastric feeding tubes that have side apertures provided a predetermined distance from the tube distal end that allow liquid to pass between the lumen and the body cavity.

In the description, reference is made to a tube distal portion which includes a tube distal end, and a tube proximal portion which includes a tube proximal end. The tube distal portion is that portion of the tube that is advanced into the patient's body and the tube proximal portion is that portion of the tube that remains external to the patient's body. The tube proximal end is adapted to couple with apparatus suitable for which the medical tube is used.

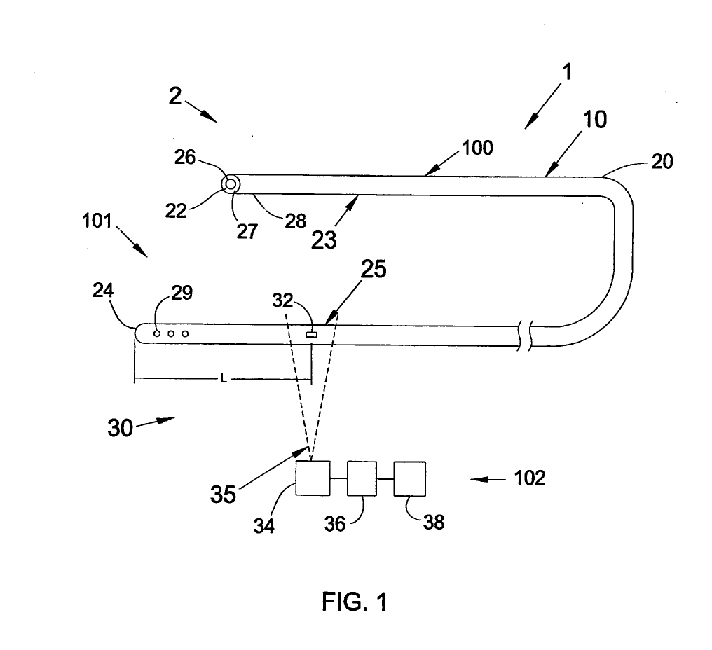

FIG. 1 is a side perspective view of a system 1 including a position-sensing medical tube 100 and sensor electronics 102, in accordance with an embodiment of the present invention. The position-sensing medical tube 100 comprises a medical tube 20 and position-sensor apparatus 101 coupled to the medical tube 20 in a predetermined location. The position-sensor apparatus 101 is adapted to communicate with the sensor electronics 102 so as to provide information dependent on the relative position of the medical tube 20. Movement of the medical tube 20, and thus the coupled position-sensor apparatus 101 is sensed by the sensor electronics 102. The sensor electronics 102 interprets state-data as that the medical tube 20 has moved and responds in a predetermined way, such as, but not limited to, triggering an alarm and turning off a process.

Referring again to FIG. 1, system 2 is provided wherein the position-sensing medical tube 100 is an RFID-tagged medical tube 10 comprising a medical tube 20 and wherein the position-sensor apparatus 101 is an RFID (radio-frequency identification) tag 32 coupled thereto, and the sensor electronics 102 is a radio-frequency sensor electronics 30 including an RFID sensor element 34, data communication electronics 36, and receiving electronics 38, in accordance with an embodiment of the present invention. The medical tube 20 is an elongated tubular member comprising a tube proximal end 22, a tube distal end 24, and a lumen 26 therebetween. The medical tube 20 further comprises a tube wall 27 defined by the lumen 26 and a tube outer surface 28. The medical tube 20 further comprises a tube distal portion 25, including the tube distal end 24. The tube distal portion 25 is that portion of the medical tube 20 that typically resides within the patient 40 when in use. The medical tube 20 further comprises a tube proximal portion 23, including the tube proximal end 22. The tube proximal portion 23 is that portion of the medical tube 20 that typically resides outside of the patient 40 when in use.

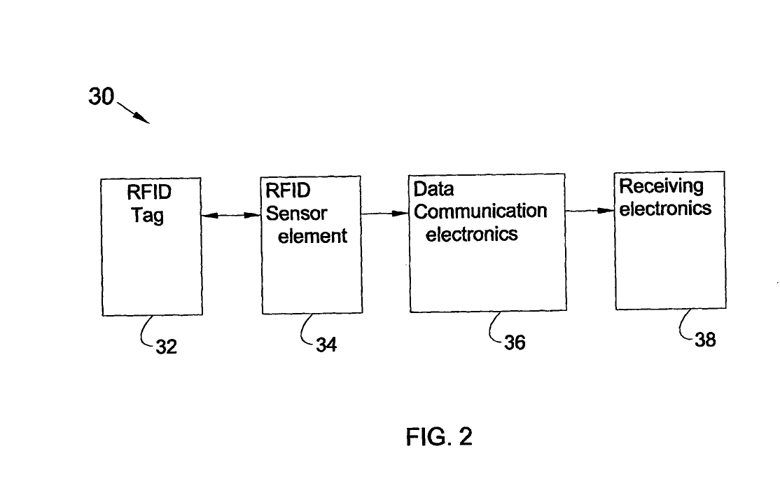

FIG. 2 is a schematic diagram of the radio-frequency sensor electronics 30 comprising an RFID sensor element 34, data communication electronics 36, and receiving electronics 38 adapted to communicate with one or more radio-frequency identification (RFID) tags 32, in accordance with an embodiment of the present invention. RFID tags 32 are known in the electronics arts, such as those described in US Patent Application Publication US 2003/0179078, and in U.S. Pat. No. 6,700,931. An RFID tag 32 is a small object that contains electronics including an antenna to enable it to receive and respond to radio-frequency queries from an RFID sensor element 34. The RFID sensor element 34 communicates data received from the RFID tag 32 to the data communications electronics 36 which in turn communicates the data to the receiving electronics 38.

Referring again to FIG. 1, an RFID tag 32 is coupled to the medical tube 20 in a predetermined location of the tube distal portion 25. The RFID tag 32 is located a predetermined distance L from the tube distal end 24 suitable for the particular purpose. The RFID tag 32 is at a predetermined distance from the tube distal end 24 such that when the medical tube 20 is properly positioned within the patient's body, the RFID tag 32 is located within the patient's body. The RFID sensor element 34 is positioned in, on, or near, to the patient in such a way as to detect the RFID tag 32. When the medical tube 20 becomes displaced from its proper placement, the RFID tag 32 moves relative to the RFID sensor element 34. This movement of the RFID tag 32 is sensed by the RFID sensor element 34, which in turn communicates state-data to the data communication electronics 36, and to the receiving electronics 38. The receiving electronics 38 interprets the state-data as that the medical tube 20 has moved and responds in a predetermined way, such as, but not limited to, triggering an alarm and turning off a process.

FIG. 2 shows schematically an RFID sensor element 34 in accordance with an embodiment of the present invention. The RFID sensor element 34 comprises an antenna array adapted to sense an RFID tag 32 as is known in the art. The RFID sensor element 34 is coupled to data communication electronics 36 adapted to communicate with the receiving electronics 38. The data communication electronics 36 provides power and a communication means for communicating between the RFID sensor element 34 and the receiving electronics 38. Wired and/or wireless communication electronics are included so as to communicate data from the RFID sensor element 34 to the receiving electronics 38. The wireless communication electronics is adapted to allow for remote positioning of the receiving electronics 38, such as, but not limited to, a bedside, a nursing station, and to a computer network.

The antenna of the RFID sensor element 34 is adapted to provide a sensor volume 35 within which an RFID tag 32 is detected, as is known in the art, as shown in FIG. 1. An RFID tag 32 that lies within the sensor volume 35 is caused to produce a radio-frequency signal that is received by the RFID sensor element 34. The RFID sensor element 34 communicates the received signal to the data communications electronics 36 which communicates a signal to the receiving electronics 38. State data, as used herein, refers to any signal communicated to the receiving electronics 38 by the data communication electronics 46. Such state data includes, but is not limited to, null signal that indicates that no RFID tag 32 is within the sensor volume 35; a received signal that indicates that an RFID tag 32 is within the sensor volume 35; and identification data received from a particular RFID tag 32 that indicates that that a particular RFID tag 32 is within the sensor volume 35.

The size of the sensor volume 35 is predetermined for a particular purpose. In an embodiment, the sensor volume 35 is sufficiently small so as to detect an individual RFID tag 32 on an RFID-tagged medical tube 12 comprising multiple RFID tags 32 distributed along the medical tube length. In another embodiment, the sensor volume 35 is sufficiently small so as to detect the movement of an individual RFID tag 32 associated with harmful displacement of the RFID-tagged medical tube 12 within the patient, but sufficiently large to prevent movement of the RFID tag 32 either within or out of the sensor volume 35 due to non-critical physiological movements, such as, but not limited to movement associated with respiration.

The receiving electronics 38 comprises circuitry and/or apparatus suitable for a particular purpose. It is appreciated that the receiving electronics 38 can be configured for many purposes in response to receiving state data from the RFID sensor element 34. Such purposes include, but not limited to, cutting power to a pump, activating a valve, activating a switch, activating a timing circuit, and activating an alarm. It is appreciated that the receiving electronics 38 can comprise controls suitable for a particular purpose. Such controls include, but are not limited to, sensitivity calibration, recalibration at suitable time intervals, trigger delay, among others. It is appreciated that the receiving electronics 38 can be configured to provide one or a combination of purposes and controls.

In accordance with an embodiment of the present invention, the receiving electronics 38 comprises circuitry so as to stop a process or trigger a mechanism. By way of example, wherein the RFID-tagged medical tube 10 is used to deliver enteral nutrition, the receiving electronics 38 may be configured to activate a valve so as to shut off delivery of the enteral nutrition to the patient upon receiving a signal that the RFID-tagged medical tube 10 has become displaced. In another embodiment, the mechanism and an audio and/or visual alarm are used in combination to stop a process and to notify a health care worker or patient when the receiving electronics 38 receives a signal that the RFID-tagged medical tube 10 has become displaced.

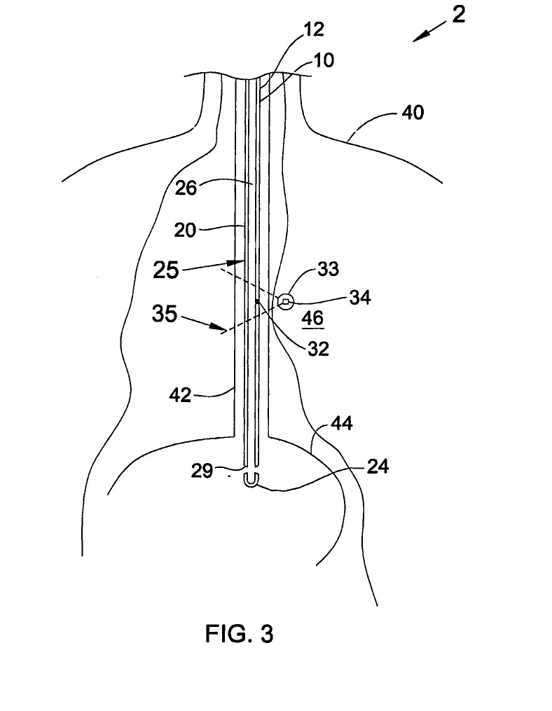

FIG. 3 is a front partial cut-away view showing a medical tube system 2 comprising an RFID-tagged medical tube 10 and an RFID sensor element 34 relative to a patient 40, in accordance with an embodiment of the present invention. In the following description, the medical tube 10 is used as a nasogastric tube used for enteral feeding by way of example, but is not limited thereto. The RFID-tagged medical tube 10 comprises an RFID tag 34 coupled to the tube distal portion 25. The RFID-tagged medical tube 10 further comprises a lumen 26 through which enteral nutrition is delivered from an outside source to the patient's stomach 44 or duodenum. The RFID-tagged medical tube 10 comprises a plurality of apertures 29 about the tube distal portion 25 that permit enteral nutrition to pass from the lumen 26 into the stomach 44.

In an embodiment in accordance with a method of the present invention, an RFID-tagged nasogastric tube 12 is positioned within the patient 40 by passing the tube distal end 24 through the nose or oral pathway, through the esophagus 42 and disposed in either the stomach 44 or duodenum. The RFID sensor element 34 is placed on a relatively stationary portion of the patient 40, such as, but not limited to, the chest 46, at a predetermined location and positioned relative to the RFID tag 32 such that the RFID tag 32 is within the sensor volume 35 and detected by the RFID sensor element 34. This establishes a baseline condition where the RFID-tagged medical tube 10 is properly placed. In the circumstance where the RFID-tagged medical tube 10 is displaced or pulled out of the esophagus 42 a predetermined distance, the RFID tag 32, being coupled to the RFID-tagged medical tube 10, will move relative to the sensor volume 35 defined by the RFID sensor element 34. Where the change in relative position is great enough to move the RFID tag 32 out of the sensor volume 35, the RFID sensor element 34 communicates a signal to the data communication electronics 36 which in turn communicates the signal to the receiving electronics 38. The receiving electronics 38, upon receipt of the signal that the RFID tag 32 is no longer detected by the RFID sensor element 34, interprets the signal and commences an event, such as, but not limited to, trigger an alarm and halt the delivery of enteral feeding to the patient.

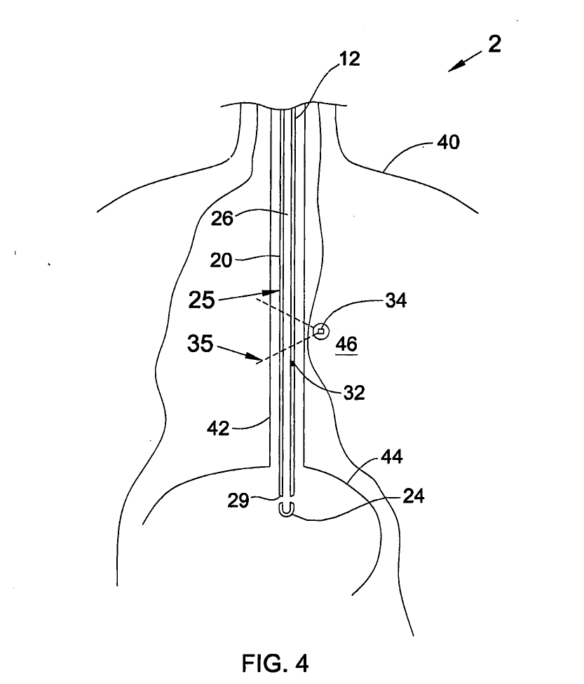

FIG. 4 is a front partial cut-away view showing a medical tube system 2 comprising an RFID-tagged medical tube 10 and an RFID sensor element 34 relative to a patient 40, in accordance with an embodiment of the present invention. In an embodiment in accordance with another method of the present invention, the RFID-tagged medical tube 10 is properly positioned through the esophagus 42 of a patient 40. The RFID sensor element 34 is placed exterior to the patient 40, such as, but not limited to the chest 46, at a predetermined location and positioned relative to the RFID lag 32 such that the RFID tag 32 is not within the sensor volume 35 and not detected by the RFID sensor element 34. More particularly, the sensor volume 35 of the RFID sensor element 34 is located to be aligned with and distal to the RFID tag 32 such that in the circumstance where the RFID-tagged nasogastric tube 12 is displaced or pulled out of the esophagus 42 a predetermined distance, the RFID tag 32, being coupled to the RFID-tagged medical tube 10, will move into the sensor volume 35. This establishes a baseline condition where the RFID-tagged medical tube 10 is properly placed. The RFID sensor element 34 is adapted to detect the RFID tag 32 having moved within the sensor volume 35 and communicate a signal to the data communication electronics 36 which in turn communicates the signal to the receiving electronics 38. The receiving electronics 38, upon receipt of the signal that the RFID tag 32 has moved into the sensor volume 35 interprets the signal and commences an event, such as, but not limited to, trigger an alarm and halt the delivery of enteral feeding.

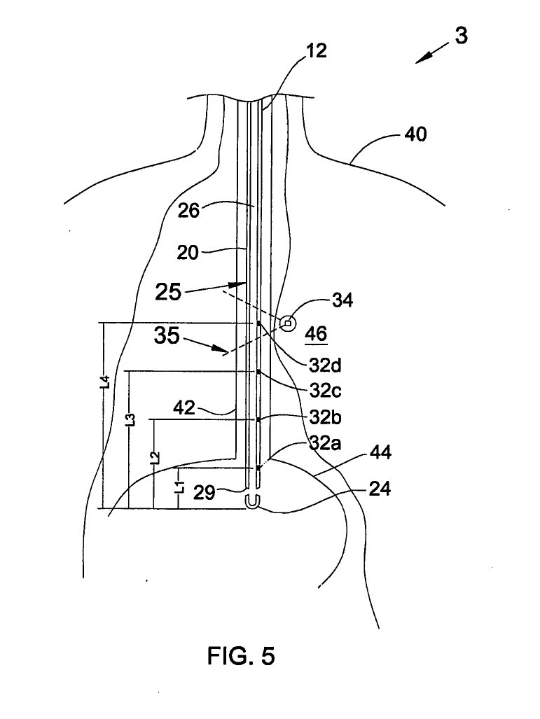

FIG. 5 is a front partial cut-away view showing a medical tube system 4 comprising an RFID-tagged medical tube 10 and an RFID sensor element 34 relative to a patient 40, in accordance with an embodiment of the present invention. The RFID-tagged medical tube 10 comprises a plurality of RFID tags 32a,b,c,d sequentially placed along the tube length. In other words, each RFID tag 32 is located at increasingly further distances with respect to the nasogastric tube distal end 24. A first RFID tag 32a is a first distance L1 from the tube distal end 24, a second RFID tag 32b is a second distance L2 greater than L1 from the tube distal end 24, and so forth, for an N-number of RFID tags 32.

The amount of movement of the RFID-tagged medical tube 10 sensed by the RFID sensor element 34 is determined, among other things, by both the number of RFID tags 32 associated with the medical tube and the distance between each successive RFID tag 32. For example, the greater the number of RFID tags 32 and the smaller the distance between each successive RFID tag 32 results in less movement required by the medical tube 10 to cause an RFID tag 23 to enter or exit the sensor volume 35 providing a greater sensitivity of movement by the RFID sensor element 34 to a change in position of the RFID-tagged medical tube 10 in relationship to the patient 40.

In an embodiment in accordance with another method of the present invention, an RFID sensor element 34 is placed on the patient 40, such as, but not limited to the chest 46, at a predetermined location and positioned relative to the RFID tags 32 such that a fourth RFID tag 32d is within the sensor volume 35 and detected by the RFID sensor element 34. This establishes a baseline condition where the RFID-tagged nasogastric tube 12 is properly placed.

In the circumstance where the RFID-tagged medical tube 10 is displaced in the esophagus 42 a first predetermined distance, a fourth RFID tag 32d moves out of the sensor volume 35 defining a first state condition. The RFID sensor element 34 communicates a signal to the data communication electronics 36 which in turn communicates the signal to the receiving electronics 38. The first state condition is associated with movement of the RFID-tagged medical tube 10 sufficient to warrant review and possibly repositioning, but does not present a significant risk to the patient.

In the circumstance where the RFID-tagged medical tube 10 is further displaced or pulled out of the esophagus 42 to a second predetermined distance 14 minus L3, the third RFID tag 32c moves into the sensor volume 35 defining a second state condition. The RFID sensor element 34 communicates a signal to the data communication electronics 36 which in turn communicates the signal to the receiving electronics 38. The second state condition is associated with movement of the RFID-tagged medical tube 10 sufficient to warrant immediate intervention. The receiving electronics 38, upon receipt of the signal that the third RFID tag 32c has moved into the sensor volume 35 interprets the signal and commences an event, such as, but not limited to, trigger an alarm and halt the flow of enteral feeding.

Providing more than one RFID tag 32 also provides more choices as to placement of the RFID sensor element 34. If one of the RFID tags 32 is positioned where it would be inconvenient to locate the RFID sensor element 34 adjacent thereto, then it is likely that another RFID tag 32 will be provided in a more convenient location.

It is appreciated that embodiments of the present invention may comprise RFID-tagged medical tubes having one or more RFID tags associated with one or more RFID sensor elements, suitable for a particular purpose. A single RFID tag and a single RFID sensor element is suitable for detecting whether the RFID tag on the RFID-tagged medical tube has moved into or out of the sensor volume defined by the sensor element. However, an RFID-tagged medical tube having a plurality of RFID tags disposed sequentially along the length of the RFID-tagged medical tube provides the benefit of identifying multiple RFID tags providing additional positional information related to the medical tube. Further, providing more than one RFID sensor element in conjunction with an RFID-tagged medical tube having one or more RFID tags provides for additional positional information related to the medical tube, as well as providing redundancy. Therefor, it is anticipated that a system comprising an RFID-tagged medical tube may include one or more RFID tags and one or more RFID sensor elements, suitable for a particular purpose.

It is appreciated that RFID tags can provide identification information to the RFID sensor element. The RFID tag may be encoded such that a specific RFID tag may be identified by the RFID sensor element. By way of example, but not limited thereto, for an RFID-tagged medical tube having a plurality of RFID tags sequentially placed along the length of the medical tube, each RFID tag has unique identification encoding. As each RFID tag enters the sensor volume, the RFID sensor element can identify which of the plurality of RFID tags is within the sensor volume. Beneficial information regarding the position of the RFID-tagged medical tube can be gathered based on which particular RFID tag is within the sensor volume at a particular time.

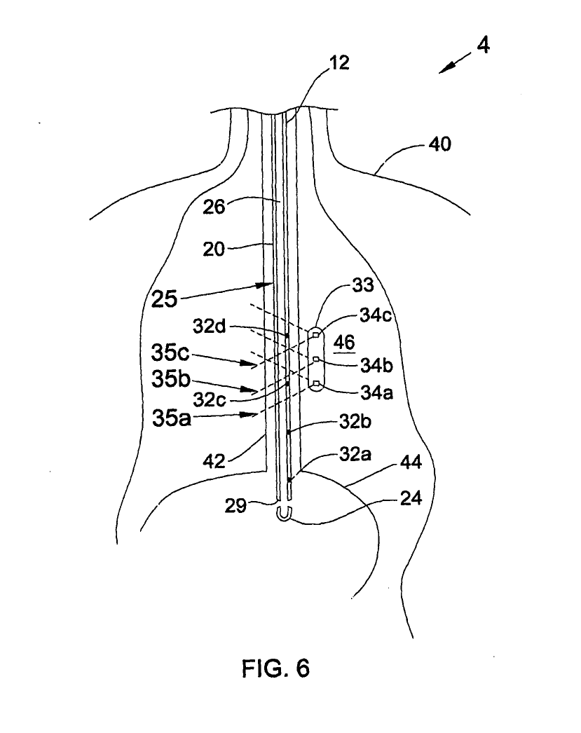

FIG. 6 is a front partial cut-away view showing a medical tube system 6 comprising an RFID-tagged medical tube 10 and a plurality of RFID sensor elements 34a,b,c relative to a patient 40, in accordance with an embodiment of the present invention. The RFID-tagged medical tube 10 comprises a plurality of identifiable RFID tags 32a,b,c,d sequentially placed along the tube length.

In an embodiment in accordance with another method of the present invention, the RFID-tagged medical tube 10 is properly positioned within the patient 40, and a plurality of RFID sensor elements 34a,b,c are placed on the patient 40 at predetermined locations, such as but not limited to vertically along the patient's chest 46 parallel to the esophagus 42, and positioned relative to the RFID tags 34a,b,c,d such that at least one RFID tag 32a,b,c,d is within the sensor volume 35a,b,c of at least one RFID sensor element 34a,b,c, respectively. In the circumstance where the RFID-tagged medical tube 10 is displaced or pulled out of the esophagus 42 a predetermined distance, one or more RFID tags 32a,b,c,d will move into or out of a sensor volume 35a,b,c. The respective RFID sensor element 34a,b,c communicates a signal to the data communication electronics 36 which in turn communicates the signal to the receiving electronics 38 which responds to the signal in a predetermined way.

Referring again to FIG. 3, the sensor element 34 further comprises a backing 33 adapted to removably couple the sensor element 34 to the patient 40. The backing 33 can comprise any material suitable for the particular purpose, such as, but not limited to, fabric with an adhesive coating. In an embodiment in accordance with the present invention, the backing 33 also serves as a platform to mount the RFID sensor element 34 and the data communications electronics 36 in the form of a wireless transceiver onto the patient 40.

In accordance with another embodiment, the radio-frequency sensor electronics 30 comprises a control circuit to adjust the gain of the antenna array of the RFID sensor element 34 and therefore to adjust the size of the sensor volume 35. This in turn controls the sensitivity and performance of the system. It is appreciated that the adjustment of the gain may be executed automatically by electronics-based control circuit based on which and how many of the RFID tags 32 are being sensed. The electronics may automatically, at predetermined time intervals, recalibrate itself to take into consideration such situations that may change the sensor volume 35 of the RFID sensor element 34.

In accordance with embodiments of an REID-tagged medical tube, the one or more RFID tags 32 are coupled to the medial tube 20 in a process suitable for a particular purpose. In accordance with an embodiment of the present invention, the RFID tag 32 is adhesively coupled to the tube outer surface 28 of the medical tube 20. In another embodiment, the RFID tags 32 are coupled to the tube outer surface 28 and provided with a coating overlay that redefines the tube outer surface 28 and encapsulates the RFID tags 32. In another embodiment, the RFID tags 32 are molded within the tube wall 27.

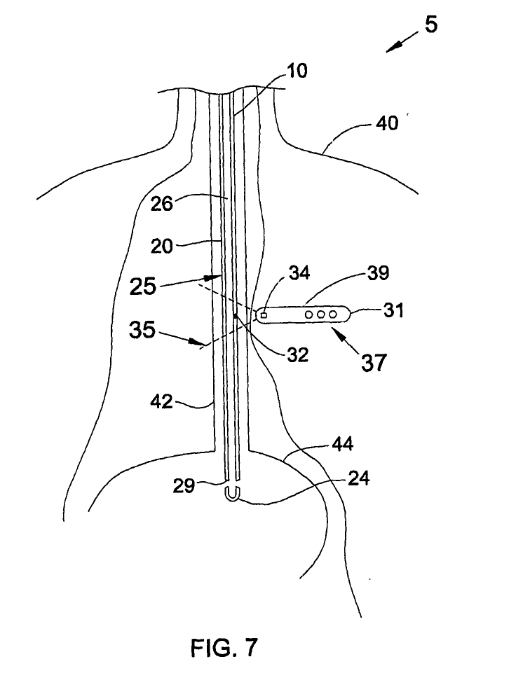

FIG. 7 is a front partial cut-away view showing a system 8 comprising an RFID-tagged medical tube 10 and a handheld RFID sensor 39 relative to a patient 40, in accordance with an embodiment of the present invention. The RFID-tagged medical tube 10 comprises an RFID tag 32 coupled to the tube distal portion 25 of the RFID-tagged medical tube 10. The handheld RFID sensor 39 comprises an RFID sensor element 34 substantially as described above contained in a housing 31 such that it may be held in an operator's hand. The handheld RFID sensor 39 comprises electronics and an indicator apparatus 37 to communicate state-data to the operator. In an embodiment in accordance with the present invention, the indicator means comprises audio and/or visual indicators of the state-data, that is, the presence and/or identification of any RFID tag 32 within the sensor volume 35 of the RFID sensor element 34.

In accordance with an embodiment of a method of the present invention, the handheld RFID sensor 39 is used to detect an RFID tag 32 associated with the RFID-tagged medical tube 10 to assist in the placement of the RFID-tagged medical tube 10 within the patient 40. By knowing the location of the RFID tag 32 in relationship to the tube distal end 24, the operator can determine the position of the RFID-tagged medical tube 10 within the patient 40 by detecting the RFID tag 32 using the handheld RFID sensor 39 positioned external to the patient.

By way of example, but not limited thereto, in an embodiment in accordance with a method of the present invention, an RFID-tagged medical tube 10, used as a nasogastric tube, comprises an RFID tag 32 located at the distal end portion 25. The RFID-tagged medical tube 10 is advanced into the patient 40. The handheld RFID sensor 39 is placed exterior to the patient 40, such as, but not limited to the abdomen, at a location where the distal end portion 25 of the RFID-tagged nasogastric tube 12 is desired to be placed. The handheld RFID sensor 39 communicates to the operator, such as with a light or sound via the indicator means 37, when the RFID tag 32 is detected. If the RFID tag 32 is not detected, the operator will know that the RFID-tagged nasogastric tube 12 was not properly placed.

The system 4 comprising an RFID-tagged medical tube 10 and handheld RFID sensor 39, in accordance with embodiments of the present invention, provides a way to detect and prevent serious medical consequences associated with the occurrence of misplacing the RFID-tagged medical tube 10 within the patient. By way of an example associated with nasogastric tubes used for enteral feeding, the system 8 of the present invention provides a way to detect and prevent serious medical consequences associated with misplacing a nasogastric tube distal end into the patient's lungs where the tube distal end was to be placed within the patient's stomach. Another example is to prevent serious medical consequences associated with misplacing the nasogastric tube distal end into the patient's esophagus where the tube distal end was to be placed within the patient's stomach or duodenum.

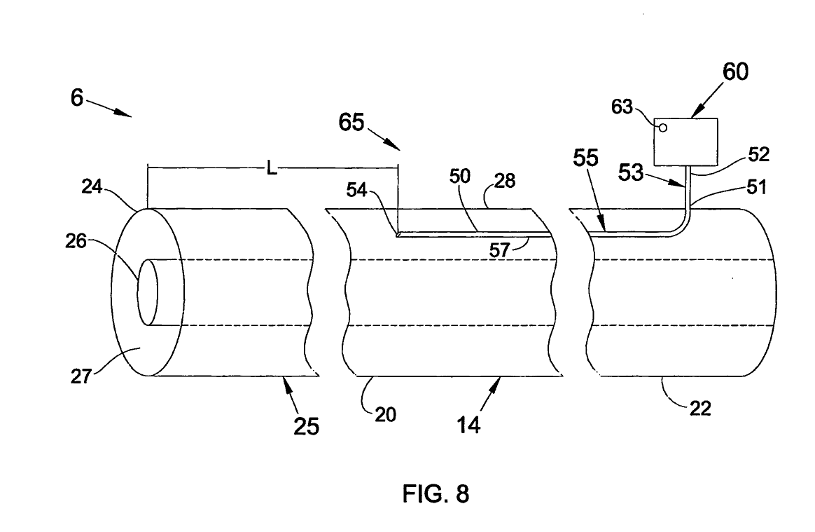

FIG. 8 is a side view of a system 6 wherein the position-sensing medical tube 100 of FIG. 1 is an optical-tagged medical tube 14 comprising a medical tube 20, and wherein the position-sensor apparatus 101 is a light sensor apparatus 65 coupled thereto, and the sensor electronics 102 is sensor electronics 60, in accordance with an embodiment of the present invention. The light sensor apparatus 65 is adapted to detect light and transmit a signal based on the intensity of the light to the sensor electronics 60. The system 6 operates on the premise that the amount of available light is greater outside than inside a patient's body. When the optical-tagged medical tube 14 is properly placed within the patient's body, the light sensor apparatus 65 detects a first intensity of light, whereas when the optical-tagged medical tube 14 is displaced from the proper position, the light sensor apparatus 65 detects an increased intensity of light. Sensor electronics 60 is adapted to detect the intensity of the light received by the light sensor apparatus 65 and provide a response suitable for a particular purpose. The light that is detected by the light sensor apparatus 65 may come from one or more sources outside of the patient, such as, but not limited to, ambient room light and a dedicated light source.

It is appreciated that various apparatus may be used to provide the function of the light sensor apparatus 65, including, but not limited to, optical fiber and electronic photodetector. The light sensor apparatus 65 has the property of being able to couple with the medical tube 20 and not substantially interfere with the function of the medical tube 20. The light sensor apparatus 65 is adapted to communicate with the sensor electronics 60 that is adapted to accept the signal from the light sensor apparatus 65 and respond to the signal in a predetermined way.

Referring again to FIG. 8, the light sensor apparatus 65 comprises a light guide (LG) 50, in accordance with an embodiment of the present invention. Light guide (LG) 50 refers to any optical component that transmits light in a preferential predetermined way. An example of a LG 50 is, but is not limited to, an individual or bundle of optical fibers adapted to transmit light along its length from one end to the other. The LG 50 may also be a material property of the medical tube that is adapted to transmit light. The LG 50 comprises a LG proximal end 52 and a LG distal end 54.

The LG 50 further comprises a LG distal portion 55, including the LG distal end 54. The LG distal portion 55 is disposed on the tube outer surface 28. The LG 50 further comprises a LG proximal portion 53, including the LG proximal end 52. The LG proximal portion 53 is not coupled to the tube outer surface 28 so as to be coupled with the sensor electronics 60. The LG proximal end 52 is adapted to couple with the sensor electronics 60 which will be further described below. The sensor electronics 60 is adapted to detect received light from the LG 50, among others.

Referring again to FIG. 8, in accordance with an embodiment of the present invention, the LG 50 comprises an optical fiber 51 with the physical property that light can only enter the LG distal end 54 and not along the LG outer surface 57 of the optical fiber 51. It is appreciated that the term optical fiber used herein refers to a single optical fiber as well as a bundle of optical fibers having common termini. The optical fiber 51 can be either individually, or as a bundle, provided with a jacket or coating of opaque material so as to prevent light from entering the LG outer surface 57. The jacket may be comprised of, but not limited to, one or more layers of polymer, PVC, or Fluoride Co-Polymer suitable for the particular purpose. The LG distal end 54 is located a predetermined distance L from the tube distal end 24 suitable for a particular purpose. The LG distal end 54 is at a predetermined distance from the tube distal end 24 such that when the medical tube 20 is properly positioned within the patient's body, the LG distal end 54 is located within the patient's body and therefore receives and transmits a reduced intensity of light to the LG proximal end 52. When the medical tube 20 becomes displaced from its proper placement by an amount that is clinically significant, the LG distal end 54 moves out of the patient and receives an increased intensity of light which is detected by the sensor electronics 60 via the LG proximal end 52. The sensor electronics 60 interprets the optical signal as that of the medical tube 20 having moved and responds in a predetermined way, such as, but not limited to, triggering an alarm and turning off a process.

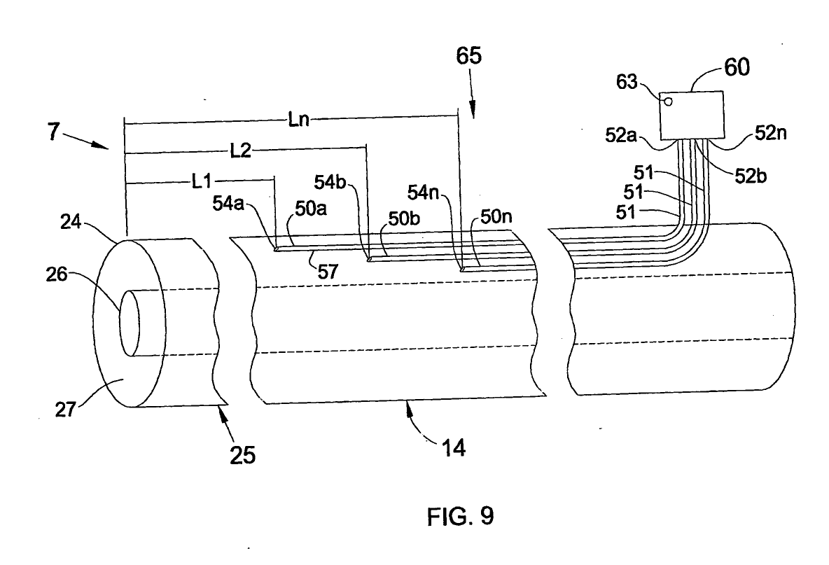

FIG. 9 is a side view of a system 7 including an optical-tagged medical tube 14 comprising a medical tube 20, light sensor apparatus 65, and sensor electronics 60, in accordance with an embodiment of the present invention. The light sensor apparatus 65 comprises a plurality of LGs 50a,b,n comprising optical fibers 51. The LG distal ends 54a,b,n of each LG 50a,b,n are located at different predetermined distances L1, L2, Ln from the tube distal end 24 suitable for the particular purpose. The LG distal ends 54a,b,n are at a predetermined distance from the tube distal end 24 such that when the optical-tagged medical tube 14 is properly positioned within the patient's body, one or more of the LG distal ends 54a,b,n are located within the patient's body and therefore receives and transmits a reduced intensity of light to the LG proximal end 52a,b,n. When the optical-tagged medical tube 14 becomes displaced from its proper placement, one or more of the one or more LG distal ends 54a,b,n previously within the patient moves out of the patient and receives an increase in the intensity of light which is detected by the sensor electronics 60 via the LG proximal ends 52a,b,n. The sensor electronics 60 interprets the optical signal(s) as that of the optical-tagged medical tube 14 having moved and responds in a predetermined way, such as, but not limited to, triggering an alarm and turning off a process.

The amount of movement of the optical-tagged medical tube 14 sensed by the sensor electronics 60 is determined, among other things, by both the number of LG distal ends 54a,b,n associated with the medical tube and the distance between each successive LG distal end 54a,b,n. For example, the greater the number of LG distal ends 54a,b,n and the smaller the distance between each successive LG distal ends 54a,b,n results in a greater sensitivity of movement sensed by the sensor electronics 60 to a change in position of the optical-tagged medical tube 14 in relationship to the patient 40.

In an embodiment in accordance with the present invention, the distance between each successive LG distal ends 54a,b,n is sufficiently small so as to allow detection of harmful displacement of the medical tube 20 within the patient, but sufficiently large to prevent an increase of light intensity due to non-critical physiological movements, such as, but not limited to movement associated with respiration.

In an embodiment in accordance with the present invention, at least one LG distal end 54 is outside of the patient and exposed to the available light. The signal provided by this LG 50 is used for a number of purposes, including, but not limited to, to calibrate the system and to warn that no light is available to allow for proper operation of the system. In an embodiment, when the LG 50 that is external to the patient detects no light, the sensor electronics 60 interprets the no-light signal as a system fault and responds in a predetermined way, such as, but not limited to, triggering an alarm and turning off a process.

In another embodiment in accordance with the present invention, the LG 50 comprises an optical fiber with the physical property that light can enter the LG distal end 54 as well as along the LG outer surface 57 of the LG 50. The LG distal end 54 is located a predetermined distance L from the tube distal end 24 suitable for the particular purpose. The LG distal end 54 is at a predetermined distance from the tube distal end 24 such that when the optical-tagged medical tube 14 is properly positioned within the patient's body, the LG distal end 54 and a portion of the length of the LG 50 is located within the patient's body and therefore receives and transmits a reduced intensity of light to the LG proximal end 52. When the optical-tagged medical tube 14 becomes displaced from its intended placement, the LG distal end 54 remains within the patient, unless of course the entire medical tube 20 is removed from the patient, and an increasing amount of the LG outer surface 57 becomes external to the patient 40 and receives and transmits an increased intensity of light to the LG proximal end 52 for detection by the sensor electronics 60. The sensor electronics 60 interprets the intensity of the optical signal as that of the medical tube 20 having moved and responds in a predetermined way depending on the intensity of the received light, such as, but not limited to, triggering an alarm and turning off a process.

In an embodiment in accordance with the present invention, the sensor electronics 60 further comprises a light sensor 63 that is outside of the patient and exposed to the available light. The light sensor 63 provides a signal to the sensor electronics 60 that is used by the sensor electronics 60 to calibrate the system and provide a baseline light intensity value used to compare with that received from the LG 50. The baseline light intensity value may be used to calibrate the expected total intensity value for changes in lighting conditions. Further, wherein the light sensor 63 detects no light, the sensor electronics 60 interprets the no-light signal as a system fault and responds in a predetermined way, such as, but not limited to, triggering an alarm and turning off a process.

In another embodiment in accordance with the present invention, the optical-tagged medical tube 14 comprises at least two LGs 50 each comprising optical fiber with the physical property that light can enter the LG distal ends 54 as well as along the LG outer surfaces 57 of the LGs 50. The LG distal ends 54 are located at different predetermined distances L1, L2 from the tube distal end 24 suitable for the particular purpose. The system may be self-calibrating to the available light by comparing the intensity of the light at the LG proximal ends 52 due to changes in available light to that due to the displacement of the optical-tagged medical tube 14. It is appreciated that the change in intensity level due to a change in available light will be different than the change in intensity level due to displacement of the optical-tagged medical tube 14. The sensor electronics 60 is adapted to detect this difference and respond accordingly. In this way the sensor electronics 60 can be programmed to compensate for lights in the patient's room being turned on and off without triggering a false tube displacement response. The sensor electronics 60 interprets the intensity of the optical signal as that of the optical-tagged medical tube 14 having moved and responds in a predetermined way depending on the intensity of the received light, such as, but not limited to, triggering an alarm and turning off a process. Wherein the sensing electronics 60 detects no light from the LG proximal ends 52, the sensor electronics 60 interprets the no-light signal as a system fault and responds in a predetermined way, such as, but not limited to, triggering an alarm and turning off a process.

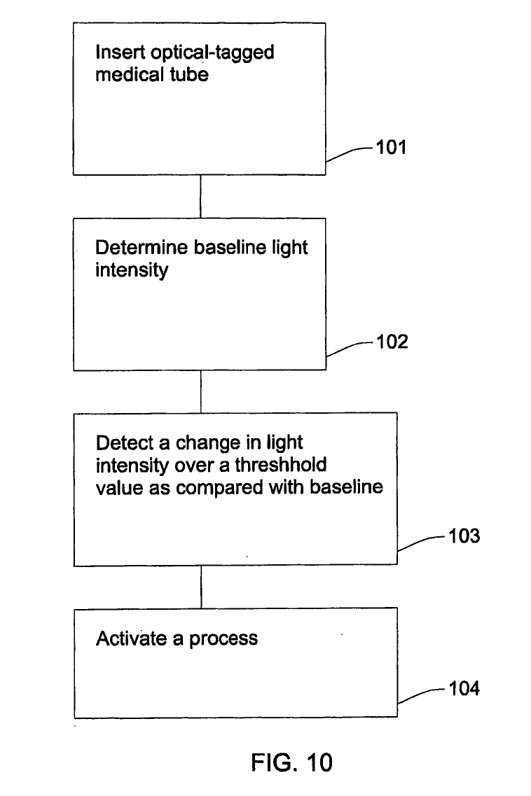

FIG. 10 is a flow chart of a method in accordance with an embodiment of the present invention. The optical-tagged medical tube is inserted into the patient to the proper position 101; a baseline light intensity detected by the system is determined 102; a change in light intensity over a threshold value as compared with the baseline is detected 103; and a process is activated in response to the change in light intensity 104.

The sensor electronics 60 may be adapted to accommodate for the daily fluctuations in ambient lighting conditions, such as associated with the turning on additional room lights or the light intensity fluctuation from night to day.

In an embodiment in accordance with the present invention, the sensor electronics 60 comprises a light sensor 63 that is outside of the patient and exposed to the available light. The signal provided by this light sensor 63 as shown on FIG. 8, is used for a number of purposes, including, but not limited to, to calibrate the system and to warn that no light is available to allow for proper operation of the system. In an embodiment, when the light sensor 63 detects no light, the sensor electronics 60 interprets the no-light signal as a system fault and responds in a predetermined way, such as, but not limited to, triggering an alarm and turning off a process.

In accordance with embodiments of the present invention, the LG distal portion 55 of each LG 50 is coupled to the medical tube 20 in any of a number of configurations representing various manufacturing techniques. Referring again to FIGS. 8 and 9, the LGs 50 are coupled to the tube outer surface 28, in accordance with an embodiment of the present invention. In accordance with another embodiment, the LGs 50 are overlaid or covered by a transparent material so as to protect the LGs 50.

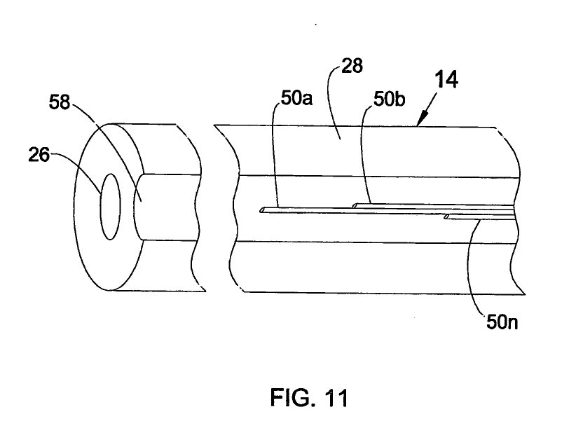

FIG. 11 is a side perspective view wherein the LGs 50 are coupled within a groove 58 extending from the tube outer surface 28, in accordance with an embodiment of the present invention.

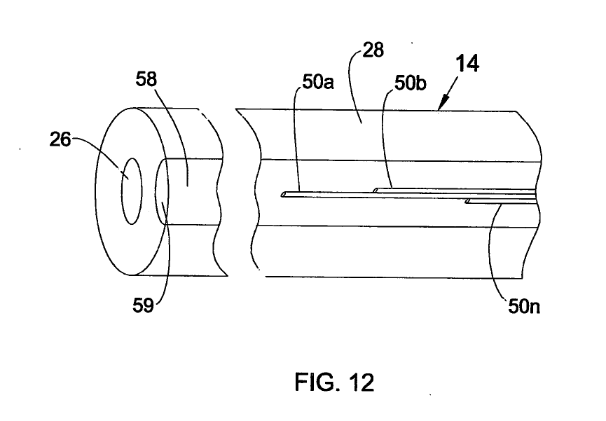

FIG. 12 is a side perspective view wherein the LGs 50 are coupled within a groove 58 extending from the tube outer surface 28, and the groove 58 is filled in with transparent material 58 to a conformal surface with the tube outer surface 28, in accordance with an embodiment of the present invention.

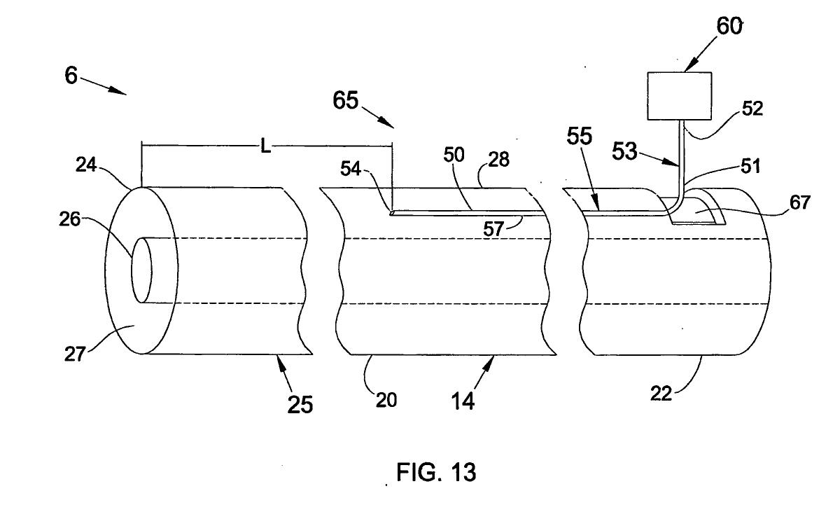

FIG. 13 is a side perspective view wherein the LG 50 is embedded in the tube wall 27 such as might be provided in a co-extrusion process, in accordance with an embodiment of the present invention. The medical tube 20 comprises a material at least partially transparent to light so that the LG may receive any available light. The LG proximal portion 53 exits the tube wall 27 via a cut-out 67 within the tube wall 27.

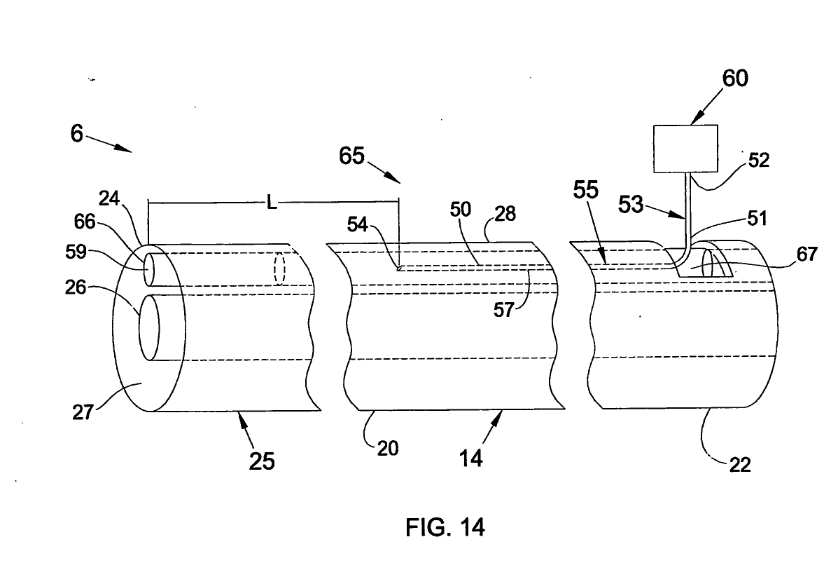

FIG. 14 is a side perspective view wherein the medical tube 20 further comprises a sensor lumen 66 that extends from the tube proximal end 23 to the tube distal end 25, in accordance with an embodiment of the present invention. The sensor lumen 66 has an inside diameter adapted to contain one or more LGs 50 therein. The sensor lumen 66 is at least partially filled and sealed with a material 59 to prevent fluid influx into the sensor lumen 66 whereby creating a lumen that extends from the tube proximal end 23 but not through to the tube distal end 25. The LG proximal portion 53 exits the sensor lumen 66 via a cut-out 67 within the tube wall 27 extending from the sensor lumen 66 to the tube outer surface 28.

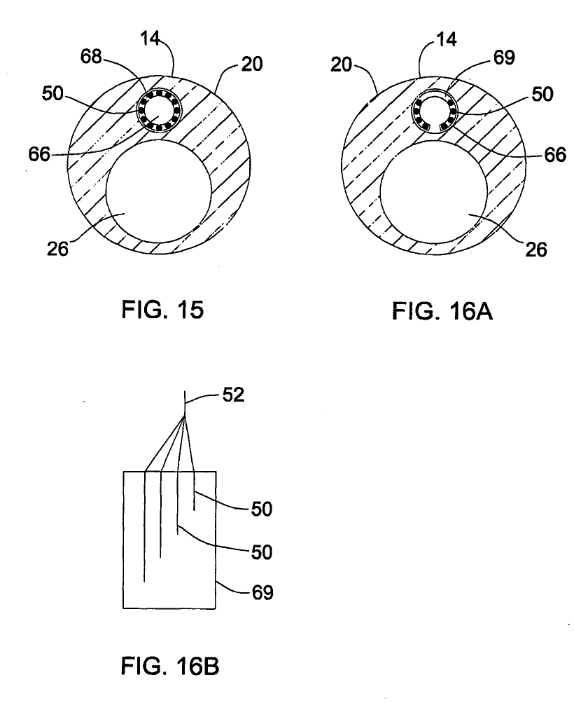

FIG. 15 is a cross-sectional view of the medical tube 20 further comprising a sensor lumen 66 that extends from the tube proximal end 23 to the tube distal end 25, in accordance with an embodiment of the present invention. The LGs 50 are coupled together by a LG tube 68. The LG tube 68 is adapted to have a diameter such that it can be coaxially placed within the sensor lumen 66. The sensor lumen 66 is at least partially filled and sealed with a material to prevent fluid influx into the sensor lumen 66, whereby creating a lumen that extends from the tube proximal end 23 but not through to the tube distal end 25. The LG proximal portion 53 exits the sensor lumen 66 either at the tube proximal end 22 or via a cut-out 67 within the tube wall 27 extending from the sensor lumen 66 to the tube outer surface 28, substantially as shown in FIG. 14.

FIG. 16A is a cross-sectional view of the medical tube 20 further comprising a sensor lumen 66 that extends from the tube proximal end 23 to the tube distal end 25, in accordance with an embodiment of the present invention. The LGs 50 are coupled together by a LG sheet 69 as shown in FIG. 16B. The LG sheet 69 is adapted to be rolled into a tubular configuration adapted to be received within the sensor lumen 66. The LG sheet is rolled to a smaller diameter than the sensor lumen and thereafter inserted into the sensor lumen to a predetermined location from the tube distal end 25. The LG sheet thereafter uncurls sufficient to place the LG sheet substantially adjacent the wall of the sensor lumen. The LG proximal ends 52 of the LGs 50 are either left separate or bundled together in a pigtail, as shown. The sensor lumen 66 is at least partially filled and sealed with a material to prevent fluid influx into the sensor lumen 66. The LG proximal portion 53 exits the sensor lumen 66 either at the tube proximal end 22 or via a cut-out 67 within the tube wall 27 extending from the sensor lumen 66 to the tube outer surface 28, substantially as shown in FIG. 14.

The LG proximal end 52 of each LG 50 is coupled to the sensor electronics 60 in a number of ways suitable for a particular purpose. In an embodiment in accordance with the present invention, each LG proximal end 52 is coupled to the sensor electronics 60 individually such that the sensor electronics 60 accepts individual signals from individual LGs 50. In this way, the sensor electronics 60 may be configured to be able to identify which of the LGs 50 is receiving an increase in intensity of light.

In accordance with another embodiment of the present invention, the LG proximal ends 52 are bundled together in a housing that branches off of the nasogastric tube, forming what is referred to as a pigtail. The individual LG proximal ends 52 remain identifiable by the sensor electronics 60. The sensor electronics 60 accepts the individual signals from individual LGs 50. In this way, the sensor electronics 60 may be configured to be able to identify which of the LGs 50 is receiving an increase in intensity of light.

In accordance with another embodiment of the present invention, the LG proximal ends 52 are bundled together in a housing that branches off of the nasogastric tube, forming what is referred to as a pigtail. The sensor electronics 60 is adapted to interpret the summation of the output of the bundle of LG proximal ends 52 to get a total intensity. The sensor electronics 60 interprets the total intensity of the optical signal and responds in a predetermined way. A light sensor 63 that is outside of the patient and exposed to the available light is used to calibrate the system and provide a baseline intensity to the total intensity value to compensate for changes in available light conditions as previously described.

The sensor electronics 60 comprises apparatus for detecting the intensity of light transmitted to the LG proximal end 52. Light-detecting apparatus are known and include, but are not limited to, semiconductor-based photodetectors. In accordance with an embodiment of the present invention, the sensor electronics 60 comprises a light sensor and alarm. Upon the light sensor detecting a predetermined change in light intensity, the alarm produces an audible, visual, or other indication, or in combination thereof, so as to alert personnel.

In accordance with another embodiment of the present invention, the sensor electronics 60 comprise apparatus so as to stop a process or trigger a mechanism, such as, but not limited to, automatically triggering a valve so as to shut off delivery of enteral nutrition to the patient. In other embodiments, the mechanism and an alarm are used in combination to stop a process and to notify a health care worker or patient when the sensor electronics 60 detects a predetermined light intensity related to the nasogastric tube having become displaced.

It is anticipated that the electronics may be configured suitable for a particular purpose. In an embodiment, the electronics are configured to simply sense a predetermined change in relative intensity of light from each LG before triggering a reaction, such as, but not limited to, triggering a mechanism or an alarm. In another embodiment, the electronics senses a predetermined change in intensity of light for a predetermined time interval before triggering a reaction. The predetermined time interval can be set to compensate for various events, such as, but not limited to, a temporary increase in ambient light. In other embodiments, the electronics is adapted to be self calibrating at predetermined time intervals, such as, but not limited to, for compensating for daytime and nighttime use.

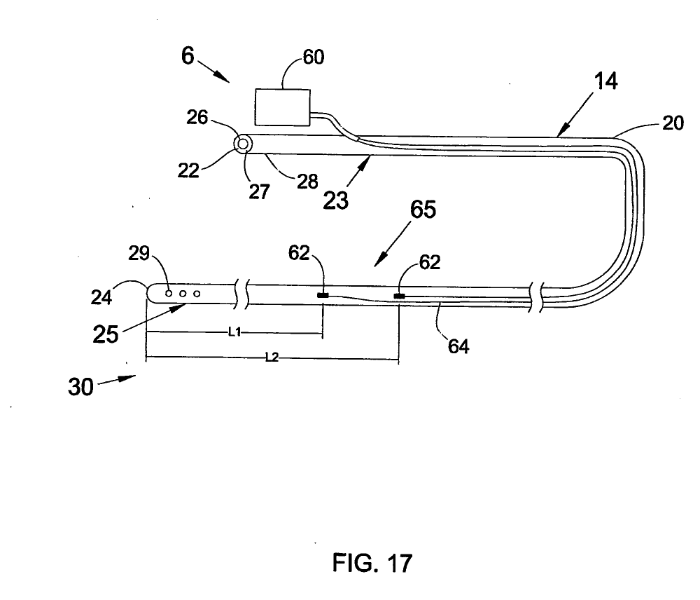

FIG. 17 is a side perspective view of a system 6 including an optical-tagged medical tube 14 comprising a medical tube 20, light sensor apparatus 65, and sensor electronics 60, in accordance with an embodiment of the present invention. The light sensor apparatus 65 comprises one or more photodetectors 62 coupled to a medical tube 20, further including sensor electronics 60, in accordance with an embodiment of the present invention. The photodetectors 62 are transducers capable of accepting an optical signal in the form of light and producing an electrical signal based on the intensity of the optical signal. An example of a photodetector 62 is a semiconductor-based photodiode. The electrical signal is communicated to the sensor electronics 60 which in turn responds to the signal in a predetermined way.

The medical tube 20 is as previously described, comprising an elongated tubular member comprising a tube proximal end 22, a tube distal end 24, and a lumen 26 therebetween. The medical tube 20 further comprises a tube wall 27 defined by the lumen 26 and a tube outer surface 28. The medical tube 20 further comprises a tube distal portion 25, including the tube distal end 24. The medical tube 20 further comprises a tube proximal portion 23, including the tube proximal end 22.

The photodetectors 62 perform a similar function as the LG distal ends 54 of the LG 50 described above. The photodetectors 62 detect light but rather than transmit the light optically to the proximal end, the photodetectors 62 convert the light to an electrical signal which is transmitted to the proximal end. As with the LG distal ends 54, the photodetectors 62 must be able to be exposed to light. Therefore, the placement and manufacturing techniques suitable for the LG's 50 are similarly suitable for the photodetectors 62.

Referring again to FIG. 17, the photodetectors 62 are coupled to the tube outer surface 28 of the medical tube 20 at predetermined locations L1 and L2, respectively, from the tube distal end 24 suitable for the particular purpose. The photodetectors 62 are at a predetermined distance from the tube distal end 24 such that when the medical tube 20 is properly positioned within the patient's body, the photodetectors 62 are located within the patient's body, and such that when the medical tube 20 becomes displaced from its proper placement a predetermined distance, at least one photodetector 62 is positioned out of the patient's body and receives an increased intensity of light. This increased intensity of light is sensed by the photodetector 62, which in turn communicates state-data in the form of an electrical signal to the sensor electronics 60. The sensor electronics 60 interprets the state-data as that of the medical tube 20 having moved and responds in a predetermined way, such as, but not limited to, triggering an alarm and turning off a process.

The photodetector 62 is coupled to the medical tube 20 in such a way so as to allow proper operation. In FIG. 17, the photodetector 62 is coupled to the tube outer surface 28 such that it will be exposed to light once outside of the patient's body. In another embodiment in accordance with the present invention, the medical tube 20 comprises a material substantially transparent to light such that the photodetector 62 may be embedded within the tube wall 27. In yet another embodiment in accordance with the present invention, the photodetector 62 is embedded within the tube wall 27 and a window of material substantially transparent to light is provided from the photodetector 62 to the tube outer surface 28 such that the photodetector 62 will detect light that is exterior to the tube outer surface 28. In yet another embodiment in accordance with the present invention, the photodetector 62 is coupled to the tube outer surface 28 which, in turn, is covered or layered with an overlay material substantially transparent to light such that the photodetector 62 will detect light exterior to the tube outer surface 28 with the photodetector 62 sealed from the environment by the overlay material.

Photodetectors commonly communicate electrical signals thorough conductive paths provided between the photodetector and associated electronics. Such conductive paths may be conductive traces and wires, among others. Photodetectors may also communicate signals wirelessly, such as through RF transmission. It is appreciated that the photodetector 62 used on the system 6 may communicate electrical signals using any suitable means. By way of example, the embodiment of FIG. 17 shows the photodetectors 62 electrically coupled to a wire 64, both of which are coupled to the tube outer surface 28. In other embodiments, a wire 64 or conductive trace embedded within the tube wall 27 is in electrical communication with the photodetector 62.

The wire 64 or conductive trace is coupled to either the sensor electronics 60 or to a wireless communicator (not shown) that provides wireless communications with the sensor electronics capable of wireless communication.

The sensor electronics 60 comprises circuitry and/or apparatus suitable for a particular purpose. It is appreciated that the sensor electronics 60 can be configured for many purposes in response to receiving state data from the photodetector 62. Such purposes include, but not limited to, cutting power to a pump, activating a valve, activating a switch, activating a timing circuit, and activating an alarm. It is appreciated that the sensor electronics 60 can comprise controls suitable for a particular purpose. Such controls include, but are not limited to, sensitivity calibration, recalibration at suitable time intervals, and trigger delay. It is appreciated that the sensor electronics 60 can be configured to provide one or a combination of purposes and controls.

Embodiments of a medical tube system of the present invention provide a medical tube that provides for monitoring whether it has become displaced which could potentially lead to serious medical conditions, such as, in the case of a nasogastric tube used for enteral feeding, aspiration of tube feeding into the patient's lungs. The displacement is made apparent to medical staff and automated systems are in place to take action, such as, in the case of enteral feeding, to shut off the delivery of enteral feeding to the patient. This allows remedial measures to be taken so that the associated morbidity and mortality can be prevented. The methods and apparatus are readily acceptable and easy to use by the medical staff, safe for the patient, and inexpensive to manufacture.

While the invention has been described in connection with specific embodiments of a nasogastric tube, embodiments where a medical tube is used for other applications is also anticipated. Any use where position of the medical tube is a concern, embodiments of the present invention may be used. Examples of such medical tubes include, but are not limited to, orogastric tubes, nasogastric tubes, percutaneous endoscopic gastrostomy tubes, percutaneous endoscopic jejunostomy tubes, endotracheal tubes, chest tubes, urinary catheters, intravenous catheters, arterial catheters, gastric decompression tubes, various suction tubes, various drainage tubes and intracranial pressure monitors.

In other embodiments, the medical tube and medical tube system are adapted to be used in applications wherein suction is used to vacate a body cavity of fluid, such as liquid and gas. Examples include, but are not limited to, stomach drainage tubes and chest tubes. In such applications, tube positioning is important to maintain proper treatment, among others. In an embodiment, when the tube is dislodged from its proper position, the position sensor apparatus detects tube dislodgement and the suction apparatus is caused to cease operation. In another embodiment, when the tube is dislodged from its proper position, the position sensor apparatus detects tube dislodgement and an alarm is activated. In another embodiment, when the tube is dislodged from its proper position, the position sensor apparatus detects tube dislodgement and an alarm is activated and the suction apparatus is caused to cease operation.

In other embodiments, the medical tube and medical tube system are adapted to be used in applications wherein respiration is used to ventilate the lungs. An example includes, but is not limited to, an endotracheal tube. In such applications, tube positioning is important to maintain proper treatment, among others. In an embodiment, when the tube is dislodged from its proper position, the position sensor apparatus detects tube dislodgement and a ventilation apparatus is caused to cease operation. In another embodiment, when the tube is dislodged from its proper position, the position sensor apparatus detects tube dislodgement and an alarm is activated. In another embodiment, when the tube is dislodged from its proper position, the position sensor apparatus detects tube dislodgement and an alarm is activated and the ventilation apparatus is caused to cease operation.

In other embodiments, the medical tube and medical tube system are adapted to be used in applications wherein intravenous intervention is used to treat a body vessel. Examples include, but are not limited to, peripheral central intravenous lines used to provide, among others nutrition, fluids, targeted medication, targeted therapy (blood thinner, anti-coagulants, clot busters, etc.) to a targeted site, such as a coronary artery, superior vena cave, among others. In such applications, tube positioning is important to maintain for proper treatment, for localized therapy, among others. In an embodiment, when the tube is dislodged from its proper position, the position sensor apparatus detects tube dislodgement and a supply apparatus is caused to cease operation. In another embodiment, when the tube is dislodged from its proper position, the position sensor apparatus detects tube dislodgement and an alarm is activated. In another embodiment, when the tube is dislodged from its proper position, the position sensor apparatus detects tube dislodgement and an alarm is activated and the supply apparatus is caused to cease operation.

In accordance with other embodiments of the present invention, the medical tube and medical tube system provide a medical tube that functions to drain a substance out of the body. This drainage can be facilitated by, for example, but not limited to, gravity and negative pressure from a vacuum system. Examples of such tubes include, but are not limited to, percutaneous endoscopic gastrostomy tubes, percutaneous endoscopic jejunostomy tubes, chest tubes, gastric decompression tubes, surgical drainage tubes, and urinary catheters. In such applications, the position of the medical tube is essential to maintain or prevent the accumulation of a substance in the body. In accordance with an embodiment, when the medical tube is displaced from its proper position, the vacuum providing negative pressure is stopped by the electronics. In another embodiment, when the medical tube is displaced from its proper position, the position sensor apparatus detects tube displacement and an audible and/or visual alarm is activated. In another embodiment, when the medical tube is displaced from its proper position, the position sensor apparatus detects medical tube displacement and an audible and/or visual alarm is activated and the negative pressure operation is stopped.

In accordance with other embodiments of the present invention, the medical tube and medical tube system provide a medical tube that functions to deliver a substance into the body. Examples of these tubes include, but are not limited to, percutaneous endoscopic gastrostomy tubes, percutaneous endoscopic jejunostomy tubes, intravenous catheters and arterial catheters. In such applications, the position of the medical tube is essential to maintain the delivery of a substance into the proper location in the body. In an embodiment, when the medical tube is displaced from its proper position, the delivery of the substance to the body is stopped by the electronics. In another embodiment, when the medical tube is displaced from its proper position, the position sensor apparatus detects tube displacement and an audible and/or visual alarm is activated. In another embodiment, when the tube is displaced from its proper position, the position sensor apparatus detects tube displacement and an audible and/or visual alarm is activated and the delivery of the substance into the body is stopped.

In accordance with other embodiments of the present invention, the medical tube and medical tube system provide a medical tube that functions to monitor the pressure exerted from inside the body. Examples of these tubes include, but are not limited to, intracranial pressure monitors. In such applications, the position of the tube is essential to maintain the monitoring of the pressure inside the body. In an embodiment, when the tube is displaced from its proper position, the monitoring of the pressure inside the body is stopped by the electronics. In another embodiment, when the medical tube is displaced from its proper position, the position sensor apparatus detects tube displacement and an audible and/or visual alarm is activated. In another embodiment, when the medical tube is displaced from its proper position, the position sensor apparatus detects tube displacement and an audible and/or visual alarm is activated and the monitoring of the pressure inside the body is stopped.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims.

1. A position-sensing medical tube system comprising:
a medical tube including an elongate tubular member having a tube proximal end and a tube distal end;
sensor electronics; and
position-sensor apparatus coupled to the medical tube in a predetermined location, the position-sensor apparatus comprising apparatus to communicate state data to the sensor electronics indicating whether the medical tube has moved from and intended location within a body to a displaced location.

2. The system of claim 1 wherein the sensor electronics comprises apparatus to respond in a predetermined way consisting of one or more of triggering an alarm and turning off a process.

3. The system of claim 1 wherein the position-sensor apparatus comprises a light sensor apparatus comprising apparatus to detect light and transmit a signal based on the intensity of the light to the sensor electronics.

4. The system of claim 3 wherein the light sensor apparatus comprises one or more light guides, the light guides having a proximal end and a distal end, the light guides having the property of accepting light from the distal end and transmitting the light to the proximal end, each light guide distal end coupled to the medical tube in a predetermined location such that one or more light guide distal ends are located within a patient when the medical tube is positioned in an intended location and one or more light guide distal ends are located outside of the patient when the medical tube is positioned in a displaced location, the sensor electronics comprising apparatus to detect the intensity of the light at the light guide proximal ends and respond in a predetermined way based on the intensity of the light.

5. The system of claim 4 wherein the light sensor apparatus comprises a plurality of light guides, each light guide distal end located a different distance from the medical tube distal end.

6. The system of claim 4 wherein each light guide is one or more optical fibers.

7. The system of claim 3 wherein the light sensor apparatus comprises one or more photodetectors having the property of converting received light into an electrical signal based on the intensity of the light, each photodetector coupled to the medical tube in a predetermined location such that one or more photodetectors are located within a patient when the medical tube is positioned in an intended location and one or more photodetectors are located outside of the patient when the medical tube is positioned in a displaced location, the sensor electronics comprising apparatus to accept the photodetector signal and respond in a predetermined way based on the photodetector signal.

8. The system of claim 3 wherein the light sensor apparatus comprises one or more light guides, the light guides having a proximal end and a distal end and defining an outer surface there between, the light guides having the property of accepting light from the distal end and the outer surface and transmitting the light to the proximal end, each light guide distal end coupled to the medical tube in a predetermined location such that one or more light guide distal ends are located within a patient when the medical tube is positioned in an intended location, the sensor electronics comprising apparatus to detect the intensity of the light at the light guide proximal ends and respond in a predetermined way based on the intensity of the light.

9. The system of claim 1 wherein the elongate tubular member comprises a tube outer surface, the position-sensor apparatus coupled at least partially to the tube outer surface.

10. The system of claim 1 wherein the elongate tubular member comprises a lumen between the tube distal end and the tube proximal end, an outer surface, and a tube wall defined by the lumen and the outer surface, the position-sensor apparatus coupled at least partially within the tube wall.

11. The system of claim 1 wherein the elongate tubular member comprises at least two lumens between the tube distal end and the tube proximal end, the position-sensor apparatus coupled at least partially within one of the at least two lumens.

12. The system of claim 1 wherein the elongate tubular member comprises at least two lumens between the tube distal end and the tube proximal end, the position-sensor apparatus at least partially coupled to a sheet, the sheet adapted to be rolled into an elongated tube and disposed at least partially within one of the at least two lumens.

13. The system of claim 1 wherein the position-sensor apparatus comprises one or more RFID (radio-frequency identification) tags and the sensor electronics comprises a radio-frequency sensor apparatus for detecting RFID tags, each of the one or more RFID tags coupled to the medical tube in a predetermined location such that one or more RFID tags are located within a patient when the medical tube is positioned in an intended location.

14. The system of claim 1 wherein the radio-frequency sensor apparatus comprises an RFID sensor element, data communications electronics, and receiving electronics, the RFID sensor element including apparatus for detecting an RFID tag and communicating a signal based on RFID tag detection to the data communication electronics, the data communication electronics including apparatus for communicating the signal from the RFID sensor element to the receiving electronics, the receiving electronics including apparatus for receiving the signal from the data communication electronics and for responding in a predetermined way based on the signal.

15. The system of claim 14 wherein the RFID sensor element communicates wirelessly with the data communications electronics, the RFID sensor element comprising apparatus for coupling to a patent adjacent the medical tube.

16. The system of claim 14 wherein the RFID sensor element comprising apparatus for coupling to a patent adjacent the medical tube.

17. The system of claim 16 wherein the RFID sensor element communicates wirelessly with the data communications electronics.

18. The system of claim 13 wherein the radio-frequency sensor apparatus comprises an RFID sensor element, a housing, and indicator apparatus, the RFID sensor element including apparatus for detecting an RFID tag and communicating a signal based on RFID tag detection to the indicator apparatus, the indicator apparatus comprising apparatus to respond in a predetermined manor, the housing suitable for containing the RFID sensor element and the indicator apparatus for handheld use.

19. The system of claim 18 wherein the indicator apparatus comprises audio and/or visual indicators triggered by the signal.

20. The system of claim 4 wherein the sensor electronics comprises apparatus to trigger an alarm in response to a predetermined signal received from the one or more light guides.

21. The system of claim 7 wherein the sensor electronics comprises apparatus to trigger an alarm in response to a predetermined signal received from the one or more photodetectors.

22. The system of claim 8 wherein the sensor electronics comprises apparatus to trigger an alarm in response to a predetermined signal received from the one or more light guides.

23. The system of claim 4 wherein the sensor electronics comprises apparatus to stop a process in response to a predetermined signal received from the one or more light guides.

24. The system of claim 7 wherein the sensor electronics comprises apparatus to stop a process in response to a predetermined signal received from the one or more photodetectors.

25. The system of claim 8 wherein the sensor electronics comprises apparatus to stop a process in response to a predetermined signal received from the one or more light guides.

26. A method of detecting movement of a medical tube from an intended location within a patient to a displaced location, comprising:
positioning a medical tube to an intended location within a patient, the medical tube including an elongate tubular member having a tube proximal end and a tube distal end, position-sensor apparatus coupled to the medical tube in a predetermined location, the position-sensor apparatus comprising apparatus to communicate state data to the sensor electronics indicating whether the medical tube has moved from an intended location within the patient to a displaced location; and
positioning the sensor electronics adjacent the position-sensor apparatus such that the position sensor electronics detects the position-sensor apparatus.
